(19) United States
(12) Patent Application Publication
(10) Pub. No.: US 2016/0317226 A1
(43) Pub. Date: Nov. 3, 2016

(54) SYSTEM AND METHOD OF COMBINED TISSUE IMAGING AND IMAGE-GUIDED LASER THERAPY

(71) Applicant: THE REGENTS OF THE UNIVERSITY OF CALIFORNIA, Oakland, CA (US)

(72) Inventors: Jared Jagdeo, Brooklyn, NY (US); Andrew Mamalis, Sacramento, CA (US)

(73) Assignee: THE REGENTS OF THE UNIVERSITY OF CALIFORNIA, Oakland, CA (US)

(21) Appl. No.: 15/146,818

(22) Filed: May 4, 2016

Related U.S. Application Data
(63) Continuation of application No. PCT/US2014/064694, filed on Nov. 7, 2014.
(60) Provisional application No. 61/902,148, filed on Nov. 8, 2013.

(51) Int. Cl. A61B 18/20; A61B 90/00

(52) U.S. Cl. CPC A61B 18/203; A61B 90/06; A61B 90/37; A61B 2018/00327

(57) ABSTRACT

A system and method for combined tissue imaging and image-guided laser therapy is provided. The apparatus has one or more imaging modalities and lasers that are controlled by a controller. The setting configuration of the laser is determined by the location of designated boundaries and the structures of the target tissue that have been imaged. Intra-operative imaging of the target tissue allows a real time evaluation of the laser administration and determines the laser configuration if the contemporary images indicate a re-treatment is necessary. Over treatment and under treatment by the laser is avoided by image-directed laser ablation or non-ablative laser therapy.

### CROSS-REFERENCE TO RELATED APPLICATIONS

This application is a 35 U.S.C. §111(a) continuation of PCT international application number PCT/US2014/064694 filed on Nov. 7, 2014, incorporated herein by reference in its entirety, which claims priority to, and the benefit of, U.S. provisional patent application Ser. No. 61/902,148 filed on Nov. 8, 2013, incorporated herein by reference in its entirety. Priority is claimed to each of the foregoing applications.

The above-referenced PCT international application was published as PCT International Publication No. WO 2015/070106 on May 14, 2015, which publication is incorporated herein by reference in its entirety.

### STATEMENT REGARDING FEDERALLY SPONSORED RESEARCH OR DEVELOPMENT

This invention was made with Government support under Grant Nos. AI080604, TR000133, and TR000134, awarded by the National Institutes of Health. The Government has certain rights in the invention.

### INCORPORATION-BY-REFERENCE OF COMPUTER PROGRAM APPENDIX

Not Applicable

### BACKGROUND

1. Technical Field

This technology pertains generally to laser surgical devices and treatment methods, and more particularly to a laser treatment device with the selection of laser treatment parameters based on the imaaing of a target tissue with an imaging modality such as Optical Coherence Tomography (OCT) or a combination of imaging modalities,

2. Background Discussion

Therapeutic laser systems that are available on the market are “blind” and the treatment of lesions (cancer, scar, or other disease processes) in organ tissues is based upon the subjective visual assessment of a physician. Clinical knowledge is often limited in the assessment of the treatment site created by ablative laser surgery of the skin. The precise depth of a skin lesion is not always clear from a clinical exam and predicting how an individual patient's skin properties will interact with ablative laser therapy is challenging and imprecise.

Therefore, the laser settings used in ablative or non-ablative laser surgeries are often selected using the laser surgeon's subjective clinical judgment, based upon a sum of prior experiences. This often results in the need for multiple treatments, or worse, the occurrence of unintended side-effects such as scarring, removal of healthy unaffected tissue, longer recovery times, post-laser procedures or additional morbidities. Because of this, there has been a rise in malpractice litigation associated with ablative laser therapy. Therefore, the need exists for the development of “smart lasers” that can enhance physician and patient outcomes.

At the present time, ablative laser therapy laser device settings are based upon 1) histology obtained from biopsying the area of skin to be treated several days or weeks in advance of the ablative laser therapy or 2) by the laser surgeon's clinical judgment, based upon physical examination findings and previous experience with other patients with similar conditions, or a combination of both approaches.

While medical laser techniques are widely used in dermatological practice to treat disease, state-of-the-art optical imaging tools are rarely used in diagnosis and surgical planning for the treatment of skin diseases. Ablative laser surgery and non-ablative therapies performed by physicians are often limited due to the inability to non-invasively image the tissue selected for laser surgical ablation or therapy and the inability to select correct laser treatment parameters or settings based upon objective criteria.

Laser settings are typically based upon the subjective clinical evaluation of the lesional skin and “treatment tables” established by laser manufacturers for various conditions, containing ranges of “acceptable” settings to treat lesional tissues. However, these limitations do not provide customized and optimized treatments for patients as each lesion and case is unique, and often requires multiple patient treatments spread over several visits and may require months or years of imprecise treatments.

There is also a need for non-invasive imaging methods to quantitate microvascular and immune responses in vivo for improved diagnosis and monitoring of a number of diseases, including port wine vascular malformations, psoriasis, skin cancer, as well as transplant rejection and graft versus host diseases. For many of these conditions, there is a need to image millimeters deep into skin tissue to assess deeper cancers and scars, as well as dysplastic or congenital nevi. The required contrast and resolution is beyond the capabilities of conventional ultrasound, necessitating the use of optical techniques.

There is also a need for more precise and accurate tailoring of ablative laser therapy to reduce side effects such as scarring and pigmentary alterations. At the same time, there is a need to immediately assess post-laser treatment to verify the complete ablation of cancerous or other types of lesions. The present technology satisfies these needs as well as others and is an overall advancement in the art.

### BRIEF SUMMARY

Ablative laser surgeries exploit the high energy and defined focal point of lasers to vaporize tissue. However, the precise depth of the skin or other tissue lesion that requires treatment is not always clear prior to surgery. The present technology is generally a system with one or more imaging modalities such as Optical Coherence Tomography (OCT) that is combined with a therapeutic/surgical laser and controller. The apparatus allows non-invasive, real time, imaging of the target tissue and then uses the information gained by imaging, such as depth, width and boundaries of the lesion, to select a laser and the configuration of the ablative laser treatment settings prior to laser surgery. The apparatus also permits the evaluation of treated sites immediately after-treatment to confirm the adequate removal of targeted lesional tissue.

The preferred surgical apparatus has a laser, at least one imaging modality, a controller such as a computer with a display and a handpiece that is configured to deliver laser light and imaging light to a target tissue. The laser can be any type of suitable ablative surgical laser or tissue treatment laser that can produce predictable results.

Preferred imaging modalities are Optical Coherence Tomography (OCT), Optical Coherence Microscopy (OCM), Photoacoustic Microscopy (PAM), and high-frequency ultrasound (HF-US) alone or in combination. Although these modalities are preferred, it will be understood that the apparatus and methods can be adapted to other imaging systems that allow real-time visualization of the treatment site.

The computer or controller preferably has programming that receives and processes data from the imaging device as an input and calculates laser settings and control commands that are based on the image data as an output. The computer or imager may also have a display to allow the user to view images and the calculated laser settings to treat the target tissue. Users may also select their own laser treatment settings guided by the imaging provided by the optical imaging system and the settings recommended by the programming.

The present technology allows a physician to non-invasively image the targeted tissue prior to laser surgery to 1) establish the boundaries and dimensions of the lesion, 2) select optimized laser settings to ablate the target lesion depending on optical properties and dimensions of the lesion, and 3) to non-invasively confirm the removal or treatment of the targeted tissues intra-operatively, and immediately re-treat if necessary.

The methods can be performed using separate imaging and ablative laser devices in sequence. The methods can also be performed simultaneously with the use of a single imaging and ablative laser combination system that images the lesional tissue, allows for the appropriate ablative laser settings to be established and configured and then ablative laser surgical treatment to be performed. Post-laser surgical follow up imaging can also be performed with the device immediately after ablative laser surgery treatment and repeated ablative laser surgery treatment performed based upon settings derived from the real-time imaging, for example.

The apparatus and methods can be used in a wide variety of applications. For example, a laser surgeon can use the apparatus to visualize and select appropriate ablative laser settings to treat skin lesions and lesional tissues from other organ systems including, oral mucosa/gingiva, ear/nose and throat, esophagus, gastrointestinal, genitourinary, ophthalmologic and cardiopulmonary treatments.

In particular, the apparatus and methods can have therapeutic uses where ablative laser therapy is used to treat various dermatological conditions such as moles, warts, photoaging, benign and malignant skin tumors, vascular lesions, skin scarring, and fibrosis. Dental conditions can also be treated including gingival hyperplasia and other lesions treatable by laser therapy. Other treatments include ear, nose and throat conditions including polyps and other lesions treatable by ablative laser therapy and ophthalmologic conditions including eye-lid neoplasms, basal cell carcinoma, papillomas and other lesions treatable by laser therapy.

Treatment of cardiopulmonary conditions, such as transmyocardial Laser Revascularization (TMR) and gastrointestinal and esophageal conditions including Barrett's esophagus and other lesions can also be performed by the apparatus and methods. Genitourinary conditions including condylomata, penile carcinoma, bladder and skin hemangiomata and other lesions can be treated by ablative laser therapy using the apparatus and methods.

According to one aspect of the technology, an apparatus is provided that has objective, real-time visualization of the treatment zone tissue structures and designation of target boundaries that provides a basis for the selection of the laser type and the appropriate ablative or non-ablative laser settings to treat the target tissue.

Another aspect of the technology is to provide a method that will allow a laser surgeon to evaluate lesions in a target tissue prior to laser therapy.

A further aspect of the technology is to provide the ability to evaluate treated sites immediately after laser treatment to confirm adequate removal of desired lesion tissue as well as evaluate post-laser treatment therapy to monitor healing and the response of the patient to the laser treatment.

Further aspects of the technology described herein will be brought out in the following portions of the specification, wherein the detailed description is for the purpose of fully disclosing preferred embodiments of the technology described herein without placing limitations thereon.

### BRIEF DESCRIPTION OF THE SEVERAL VIEWS OF THE DRAWINGS

The technology described herein will be more fully understood by reference to the following drawings which are for illustrative purposes only:

FIG. 1 is a schematic system diagram of one embodiment of the apparatus with multiple imaging modalities and laser emissions from a single handpiece.

FIG. 2 is a schematic flow diagram of one method for imaging and laser treatment with laser parameters based on the imaging according to one embodiment of the technology.

FIG. 3 is a representative OCT image (not to scale) illustrating maximal ablative width and maximal ablative depth measurements from the superficial boundary of the epidermis to the vertex of the microscopic ablative zone.

FIG. 4A is a graph of the ablative depth of a 2790-nm erbium:yttrium-scandium-gallium-garnet (Er:YSGG) laser that demonstrates a linear relationship with laser energy setting with a coefficient of determination equal to 0.99925. By comparison, a 2940-nm erbium-doped yttrium-aluminium-garnet (Er:YAG) laser (not shown) has similar light-tissue properties and has a coefficient of determination equal to approximately 1 as well.

FIG. 4B is an OCT image illustration with three different target depths. Using the linear relationship between ablation depth and laser energy, the apparatus clinician can predict the appropriate settings to use to target lesions at any point between X, Y, or Z.

### DETAILED DESCRIPTION

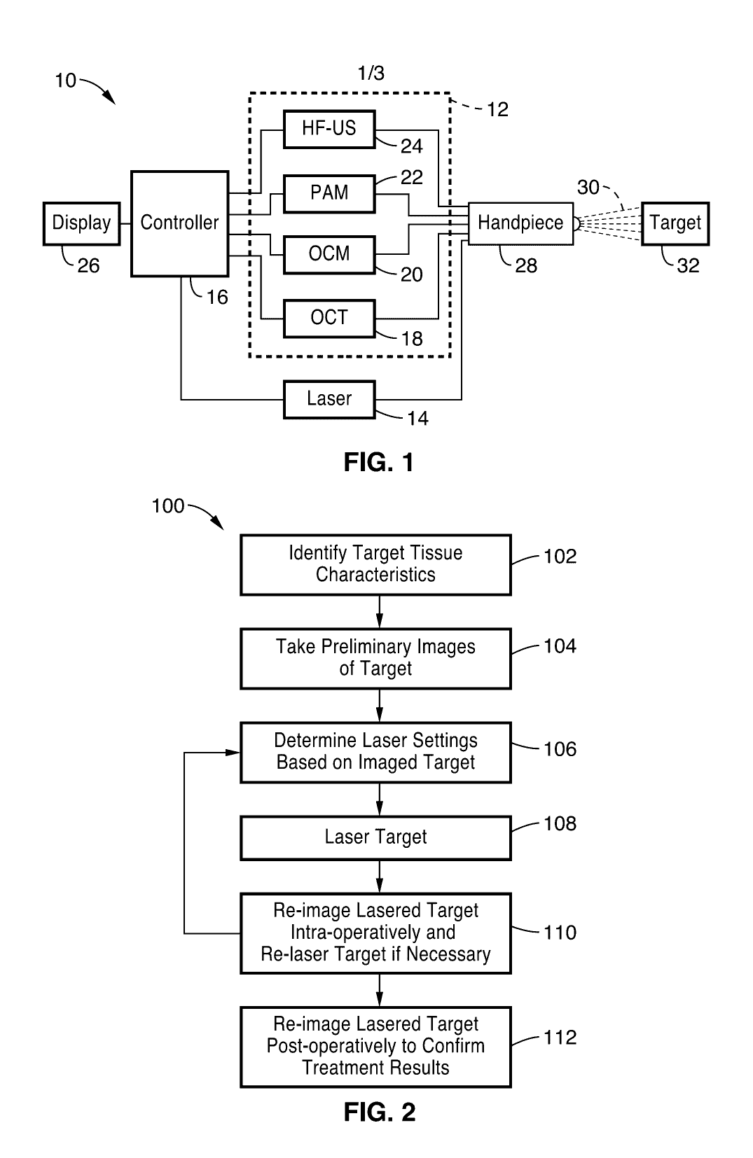

Referring more specifically to the drawings, for illustrative purposes, an embodiment of the apparatus and method for laser surgery and therapy using laser parameters based on image derived information is described and depicted generally in FIG. 1 through FIG. 4B. It will be appreciated that the methods may vary as to the specific steps and sequence and the apparatus may vary as to elements without departing from the basic concepts as disclosed herein. The method steps are merely exemplary of the order in which these steps may occur. The steps may occur in any order that is desired, such that it still performs the goals of the claimed technology.

The system is generally comprised of at least one imaging device, at least one laser device and a controller. The controller may be a dedicated circuit or a processor with programming that is integrated into either the imaging device or the laser device or as a separate control device. The controller preferably has an interface with the image devices as well as an interface with the laser delivery system so that images can be acquired, tissue locations and boundaries targeted, and laser settings calculated and configured. However, laser settings can also be set manually.

Turning now to FIG. 1, one embodiment of the apparatus 10 for laser treatment is schematically shown. The apparatus 10 generally has three interconnected modules: 1) the imaging module 12; the laser module 14 and the control module 16. Each of these modules can have different configurations and overall functions as well as provide different laser therapy or laser ablation treatment options to the user.

The imaging module 12, has at least one imaging modality. In the embodiment shown in FIG. 1, the imaging module has four imaging modalities to illustrate the variety of imaging types that can be used. Each of the imaging modalities can be used alone or in combination to provide images and target information for the apparatus 10. In one embodiment, the images of the different modalities are co-registered and viewed simultaneously.

The imaging module 12 produces images that reveal tissue micro-structures, densities, compositions and components as well as lesion dimensions and boundaries that can be targeted by the user for treatment of ablation. These characteristics can be used to determine and configure the laser settings and the nature of the laser exposure.

One particularly preferred imaging modality of the imaging module 12 is optical coherence tomography (OCT) 18. OCT imaging 18 is a rapid, non-invasive method for imaging patient skin and can identify lesional skin in the epidermis, upper and mid-dermis of patients before, during, and after treatment with fractional ablative laser therapy. OCT uses the light-based property known as low-coherence interferometry to provide rapid in vivo cross-sectional images of skin or other tissues. OCT imaging was first used clinically to take eye length measurements. In dermatology, OCT can also be used to image lesions, skin appendages, and blood vessels in the epidermal and dermal layers of the skin. OCT and the other imaging modalities can be used in the clinical assessment and therapeutic treatment of skin diseases as well as with ablative surgical procedures.

OCT imaging 18 allows rapid high resolution imaging of tissues in skin two or three-dimensions and target tissue structures; locations and boundaries can be easily identified before, during, and after laser therapy. OCT serves as an adjunctive aid to help guide ablative laser setting selections or laser therapy setting selections by allowing real-time assessment of the treatment site.

The imaging module 12 of the apparatus of FIG. 1 also has an Optical Coherence Microscopy (OCM) 20 capability. Optical coherence microscopy (OCM) 18 is a combination of confocal microscopy and optical coherence tomography (OCT) that can improve the imaging depth and contrast for cellular imaging of tissues. In one embodiment, OCT and OCM are used to produce cross-sectional images of tissue are generated based on technology referred to as the echo time-delay of light. OCT is the optical analogue of ultrasound, possessing higher resolution and lower penetration depth. Among optical microscopy techniques, OCT generally enables higher penetration depth than two-photon and confocal microscopy due to the fact that it can reject multiply scattered and out-of-focus light through a gating based on echo time delay.

Photoacoustic microscopy can add complementary absorption contrast to the in vivo OCM/OCT imaging capabilities, helping to delineate chromophores such as melanin and hemoglobin. In optical resolution Photoacoustic Microscopy (PAM) imaging 22, a short nanosecond optical pulse is focused onto the tissue. Optical absorbers (chromophores) in the tissue near the focus preferentially absorb light. When absorption occurs, heat builds up rapidly, the tissue expands locally, and ultrasound waves are generated. Excitation with different wavelengths can be used to distinguish between chromophores.

Comparable penetration depths to OCT are obtained using this method, and penetration depths are dependent on the single-pass attenuation of light. However, optical resolution PAM achieves absorption-based contrast, as opposed to scattering-based contrast in OCT. In comparison, ultrasound alone possesses excellent penetration depth, but detects signals mainly from the echo-rich dermis as opposed to echo-poor lesions of interest.

Imaging technologies in combinations such as photoacoustic microscopy (PAM) 22 and Optical Coherence Tomography (OCT) 18, based on absorption and scattering contrast, respectively, can quantify tissue chromophore and collagen content, as well as blood flow and oxygenation. Besides allowing more direct assessment of tissue status, these tools can be used to determine lesion depth in skin cancers, which is important for clinical decision making and diagnosis. Thus, besides enabling much more direct and accurate tailoring of laser treatments, new imaging technologies can greatly aid in the diagnosis and management of skin diseases.

In another embodiment, a combined Optical Coherence Microscopy (OCM) and Photoacoustic Microscopy (PAM) apparatus 22 is integrated with surgical ablation devices to enable real-time visualization of the treatment site and used to image 1 to 5 mm deep into scattering tissue, enabling the assessment of deep lesions before, during, and after treatment.

The imaging module 12 can also have a high-frequency ultrasound (HF-US) 24 capability that can be used alone or in conjunction with one or more of the other imaging modalities. HF-US imaging 24 is a technique that uses higher frequencies to yield a much improved spatial resolution by sacrificing the depth of penetration over other ultrasound techniques. It can be used in many clinical applications including visualizing blood vessel walls etc. HF-US imaging 24 can also be used to image skin cancers, benign lesions, collagen in scars or hypocollagen in chronological aging and photoaging.

The laser module 14 of the apparatus 10 of FIG. 1 has one or more laser systems that are known in the art. The laser module 14 can produce both ablative laser beams and therapeutic non-ablative laser beams. For example, laser module 14 can produce a non-ablative beam that can modify targeted tissues, such as blood vessels, hair, endogenous or exogenous tattoo pigments, melanocytic pigments, sebaceous glands, and other structures with wavelengths from 400 nm to 2000 nm in a non-ablative or minimally ablative manner. The laser module 14 can also produce ablative laser beams of 2000 nm to over 15000 nm that will permit ablative laser surgery of a tissue.

Advances in ablative laser therapy have led to the development of fractional laser skin resurfacing (FLSR) systems that emit multiple microscopic laser beams in a pixelated or grid-like pattern. The treatment zone created by fractional lasers consists of columns of ablation known as microscopic ablation zones (MAZ) and gives results approaching traditional ablative lasers with far less tissue destruction. These microscopic ablation zones stimulate dermal remodeling and neocollagenesis to reduce wrinkles, resurface skin, and reduce acne scars. Ablative laser therapy is also used to treat various other conditions such as photoaging, benign and malignant skin tumors, skin scarring, and fibrosis.

The 2790-nm erbium:yttrium-scandium-gallium-garnet (YSGG) laser is an ablative laser system that targets water to cause tissue ablation. In addition to the YSGG, other commonly used fractional ablative lasers include the 10,600-nm carbon dioxide (CO₂) and the 2,940-nm erbium:yttrium-aluminium-garnet (Er:YAG) lasers. These lasers also target intracellular water to cause tissue ablation and localized thermal damage. These ablative lasers all possess the ability to penetrate deeper than 1 mm into the skin. Although these lasers are preferred in the laser module 14, it will be understood that any type of ablative laser can be adapted for use with the apparatus 10.

The imaging module 12 and the laser module 14 are operably coupled and controlled by the control module 16. The control module 16 preferably has computer with a user interface including a display 26 and programming that sends and receives data from the imaging module 12 and calculates and controls the settings of the laser module 14. The programming of the control module 16 generally controls the parameters and settings of the laser module 14 based on the imaging results from the imaging module 12. This provides customized and optimized treatment settings for patients as each lesion and case is unique, rather than settings based on the subjective clinical evaluation of a physician and “treatment tables” established by laser manufacturers for various conditions, containing ranges of “acceptable” settings for treating lesional tissues.

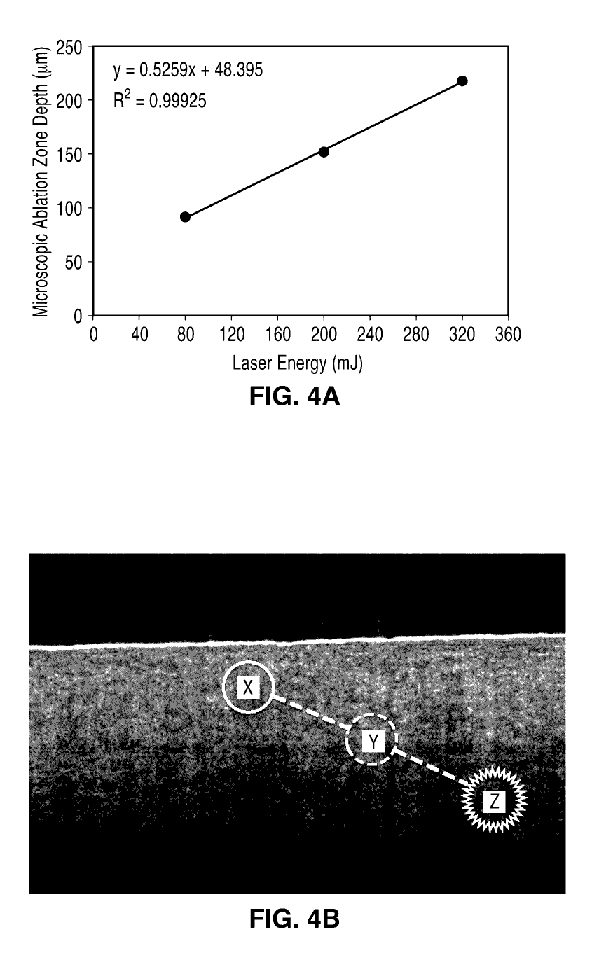

For example, the programming of control module 16 can determine the settings for the laser module 14 by accounting for the characteristics of the selected laser and the characteristics of the structures of the target tissue that is to be treated or ablated. In the case of the YSGG laser and Erbium:YAG laser, a 1:1 linear relationship between energy delivered to tissues (power or fluence) and laser penetration depth is apparent as illustrated in FIG. 4A. Therefore, the depth of the therapeutic treatment or ablation that is needed based on the imaging of the target can be determined by the programming.

In the case of a CO₂ laser, the treatment settings can be determined with algorithms that account for parameters and variations such as Fluence, as defined as laser pulse energy (joules) divided by effective focal spot area (cm²) as shown in the following formula: joules/cm².

Parameters are:

(1) How many times the laser fires repeatedly into the same spot, effectively drilling deeper into the tissue: Stacks (integer)=1,2,3,4,5.

(2) Duration of laser energy delivered to target tissues: Dwell (continuous variable)=microseconds.

(3) Power: Watts (continuous variable)=watts.

Based upon ablative laser parameters described above and other parameters, tailored algorithms for depth and width by the method of skin imaging can be derived. Examples of such algorithms are as follows:

Depth=a+a1*Stack+a2*dwell+a3*Watts
where:
   a=−140.0147
   a1=40.9649
   a2=0.1023
   a3=1.7461

Outside/Char width=b+b1*Stack+b2*dwell+b3*Watts
where:
   b=324.6398
   b1=50.9456
   b2=0.1184
   b3=2.3403

Accordingly, the energy, time of exposure, ablation depth, stacking frequency wavelength, spot size and other parameters can be calculated by the programming based on the laser, the type of treatment and locations depths of target tissues in the target structure. Programming algorithms can be defined and composed for any laser type and configuration that can produce therapeutic or ablative laser beams for use on essentially any tissue that is amenable for treatment.

The programming of the processor or controller of control module 16 can also process the data from each of the imaging modalities of the imaging module 12 and create a composite image on display 26. In one embodiment, structures and locations in the composite image can be identified by the user and displayed in a color which is different from that of surrounding tissue. The designated structures and positions can be in two or three dimensions and removal or treatment of the structures can be targeted, tracked and verified by the computer programming.

The emitters and sensors of the imaging module 12 and the laser module 14 can be assembled in a single handpiece 28 for use by the physician in the embodiment of FIG. 1. The imaging and lasing beams 30 and the sensed beams from the target 32 are emitted and sensed from one or more emitter heads in a single handpiece 28. However, in one embodiment, the individual imaging modalities of the imaging module 12 and the laser module 14 each have their own separate handpiece and the imaging and lasing are performed sequentially.

In one embodiment, the handpiece 28 has emitter heads and detectors that detect signals returning from the target tissue and produce an output that is received as input by the computer module 16 and analyzed. The detector output can also be received and processed by the imagers before being sent to the computer module 16. In response to the input, the control module 16 programming produces control instructions as an output to the laser module 14 to set (a) power, (b) exposure time, (c) pulse frequency, and (d) spot size of the laser radiation that to be emitted. Intra-operative imaging can determine the extent and boundaries of the laser treatments. And the need for re-treatment of the treatment site can be determined by intra-operative imaging once the non-ablative or ablative laser has been used to treat the targeted lesion.

Accordingly, the apparatus 10 gives the physician the ability to “see and treat” by non-invasively visualizing the boundaries of lesional tissues, for example, using OCT and then treating the lesion with laser ablation using programming, which provides customized laser settings based upon the depth of the lesional tissue assessed using OCT visualization. The device will then be able to immediately revisualize the treatment site post-laser ablation to confirm removal of lesional tissues and to re-treat the site thereafter, if necessary.

The visualization component combined with the treatment algorithms and programming would also “lower the bar” decreasing current barriers for physicians, nurses and other members of the patient's medical team that may lack extensive clinical experience with non-ablative and ablative lasers.

The procedures can also have a wider availability and use to new physicians who can visualize the depth of the lesional targets and use the pre-established programming to treat the lesion, and use of the device post-treatment to confirm the removal of lesional tissue.

The device provides a “smart laser” that can aid the operator in assessing the treatment site prior to, during and post-laser surgery, and could help limit adverse side effects and stem litigation associated with laser surgery. As medicine and therapies become more specialized and customized for individualized medical interventions for patients, this system and method will be essential for the treatment of tissues using laser therapies.

Referring now to FIG. 2, one embodiment of a method 100 of laser treatment by either therapeutic laser or laser ablation using the apparatus is set forth. At block 102, the target tissue for treatment is examined by the physician visually and with the aid of any available diagnostic devices. The characteristics and general location of the tissues to be treated are identified. For example, a skin lesion may be identified clinically and the location and nature of lesion is identified by the physician. For example, the skin thickness, collagen status—low (aging or thinning) and high (scar or fibrosis), groups of malignant or benign cells, vascularity, pigments and patient history can be identified.

At block 104, the imaging modalities of the apparatus are used to take preliminary images of the target. These preliminary images may also help to identify tissue characteristics including size and location of the boundaries of the lesion, density, vascularization and other clinically relevant lesion parameters. The preliminary imaging will also allow the designation of the target structures or tissue boundaries and the determination of the laser settings by the apparatus at block 106 that will ablate the lesion to a depth that is deep enough to remove the lesion but not too deep to cause removal of healthy tissue or to cause scarring, vascular damage and other complications.

Once the preliminary laser settings are defined by the programming of the apparatus and the laser is configured at block 106, the target tissue is lasered by the physician at block 108. The laser setting adjustments can be performed by the physician or they can be made by the apparatus automatically with confirmation by the physician.

Optionally, the treated tissue can be re-imaged immediately after the laser treatment or surgical procedure at block 110 to visualize the extent of treatment at the treatment site. The boundaries of the target portions of tissues of the target that have been removed can be imaged with the apparatus to determine whether the designated tissue has been removed. Likewise, for non-ablative procedures, the response of the targeted and treated portions of tissue can be evaluated and the boundaries between laser treated and non-treated tissues can be visualized to verify the extent of the treatments. The imagers of the apparatus can display images and composite images for review by the physician and the apparatus programming intra-operatively. If the portions of the target tissue designated for removal or treatment have not been completely removed or treated, then laser settings based on the re-imaging data can be determined at block 106. The treatment site is lasered again at block 108 and optionally re-imaged thereafter intra-operatively at block 110.

Post-operative imaging of the treatment site can also take place some period of time after the treatment procedure to confirm healing and the treatment results at block 112. This optional step at block 112 would typically take place at a follow up clinical visit.

Because the precise depth of a skin lesion is not always clear from a clinical exam, and, predicting how an individual patient's skin properties will interact with ablative laser therapy is difficult, a real-time OCT assessment of the maximal ablative depth is needed to choose appropriate ablative laser settings. The apparatus and procedures provide an objective real-time visualization of the treatment zone created by ablative or treatment lasers and enhance a laser surgeon's ability to quickly fine-tune ablative laser settings to an individual patient, lesion, or condition.

The technology described herein may be better understood with reference to the accompanying examples, which are intended for purposes of illustration only and should not be construed as in any sense limiting the scope of the technology described herein as defined in the claims appended hereto.

### EXAMPLE 1

To demonstrate OCT's capability in assessing the microscopic ablation zones (MAZ), the maximal ablative depth and width in a porcine skin model treated with fractionated YSGG laser ablation delivered at various energy levels was quantified and characterized. It was shown with OCT that there is a linear relationship between depth of YSGG fractional skin ablation and laser energy. OCT can therefore be used in conjunction with ablative lasers to assess the MAZ and that a real-time OCT assessment of the maximal ablative depth allows laser surgeons to choose appropriate ablative laser settings.

The functionality of the apparatus and methods was evaluated with either a 10,600-nm carbon dioxide (CO₂)-or a 2790-nm erbium: yttrium-scandium-gallium-garnet (Er:YSGG) laser with a VivoSight OCT machine (Michelson Diagnostics). The devices were used to analyze a test target prior to and post ablative laser therapy. A 9-inch by 9-inch section of ex-vivo porcine skin with subcutaneous fat attached was obtained from UC Davis Department of Animal Science and used as a target. The skin was from an animal that had been euthanized for other purposes within 8 hours prior to laser ablative therapy. The skin had been scalded to remove hair following euthanasia. Prior to ablative laser therapy, the skin was cleaned thoroughly with ethanol and allowed to dry.

The ex-vivo porcine skin target was treated with a 2790-nm YSGG fractional laser system (Pearl Fractional™, Cutera Inc., Brisbane, Calif.) in line pattern (density pattern #1) at energy intervals of 120, starting with the lowest device setting of 80 mJ and increasing to 200 and 320 mJ. Pearl Fractional has a spot size of 300 μm, a scan size of 10 mm×14 mm, and a pulse width of 600 microseconds. Immediately following YSGG laser treatment, OCT imaging of the microscopic ablation zone (MAZ) was performed.

A swept source OCT scanner (VivoSight; Michelson Diagnostics Ltd., UK) was used to image the MAZ created by a YSGG-FLSR (Fractional laser skin resurfacing) treatment on the target tissue. The Vivosight OCT is capable of <7.5 μm lateral resolution, <9 μm vertical resolution, and tissue penetration depths of 1.2 to 1.8 mm. The Vivosight OCT captures grayscale images with 1446×460 pixel dimensions and 4 μm pixels. The Vivosight OCT can also capture 3-dimensional images by taking multiple 2-dimensional scans using a ‘bread-slice method’ with individual sections separated by 100 μm. However, 2-dimensional images were captured and analyzed in order to reduce distortion or imaging artifacts that may result from compiling multi-scan 3-dimensional images. Due to the 3-dimensional cone-shaped nature of the MAZ, 2-dimensional cross-sectional OCT images were acquired of the maximal ablative depth and ablative width of each laser setting. These OCT scans were exported as TIFF files in order to analyze the MAZ dimensions.

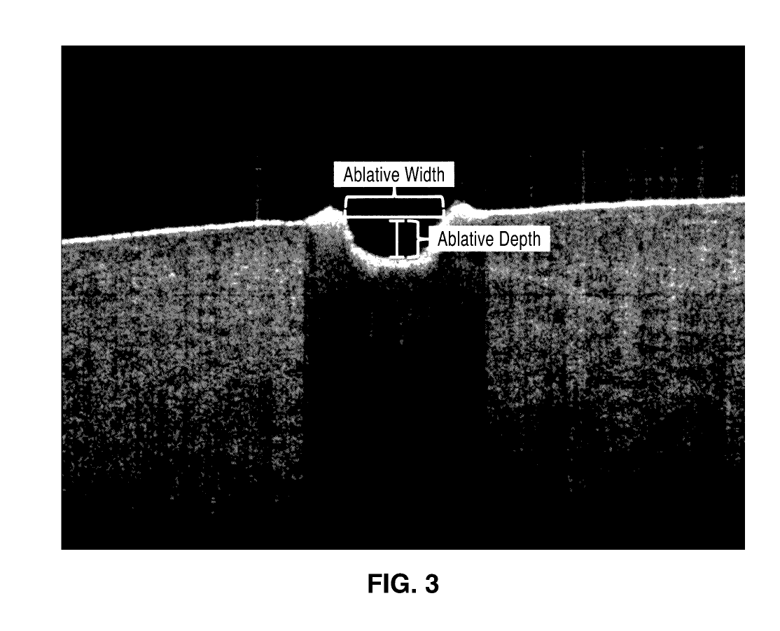

All data measurements were collected using NIH-ImageJ software version 1.47. Measurements of the maximum ablative widths and depths were taken as illustrated in FIG. 3. Mean value and standard deviation was calculated for measurements taken of each setting. Statistical analysis was performed using two-tailed Student's t-test to compare the different treatment settings. Data was plotted using Microsoft Excel for Mac 2011.

OCT images of the ablative zones induced by fractional YSGG laser therapy delivered at various energies in a porcine skin model were analyzed. The OCT measurements of the ablation zone depths demonstrated mean ablation depths of 91.5±12.5 μm at 80 mJ, 151.6±4.4 μm at 200 mJ, and 217±12.6 μm at 320 mJ. There was a significant difference observed between the mean maximal ablative depth of 80 mJ compared with 200 mJ and a significant difference between the mean maximal ablative depth of 200 mJ compared with 320 mJ (p=0.0454 and p=0.0384, respectively).

It was also observed that the treatment at each of these energy settings created a similar ablative width. The measurements of the OCT images of the ablation zone width demonstrated mean ablation widths of 336.6±40.17 μm at 80 mJ, 350.57±5.43 μm at 200 mJ, and 328.31±16.02 μm at 320 mJ. There was not a significant difference between the ablative width of 80 mJ compared with 200 mJ or 200 mJ compared with 320 mJ (p=0.753 and p=0.322, respectively).

It was shown that OCT is able to image YSGG fractional ablation zones and can be used to measure the significantly different maximal ablative depths produced by different energy settings. The OCT imaging analysis demonstrated a linear relationship between maximal ablative depth and laser energy, but not with width. Given that increasing energies produce increasing ablative depths, OCT should be a reliable alternative to histology to image the MAZ and provide real-time depth analysis.

In addition, the OCT images demonstrated a linear relationship between ablation depth and laser energy setting with a coefficient of determination equal to 0.99925 as shown in FIG. 4A. A demonstration of how OCT can be used to rapidly image patient skin and identify the depth of the target lesion skin in patients prior fractional ablative skin resurfacing is shown in FIG. 4B. If a clinician uses OCT to image a lesion at depth X, Y, or Z, they can then select the precise settings to target ablative laser therapy to that tissue depth. In addition, using the linear relationship between ablation depth and laser energy, the clinician can predict the appropriate settings to use to target lesions at any point between X, Y, or Z.

Depth of treatment is likely to be highly important in determining clinical success, however, depth is unlikely to be the singular determinant of patient outcomes. Local thermal damage surround the ablation zone plays a critical role in stimulating dermal remodeling and neocollagenesis, and excess thermal injury may contribute to adverse events. Therefore, the ability to image the thermal injury surrounding the MAZ would provide dermatologists with additional data on the laser-skin interaction and further enhance their ability to fine-tune laser settings. Unfortunately, thermally injured tissue could not be clearly defined using OCT in an ex vivo porcine model (and could be assessed using in vivo models of laser-tissue interaction) and were limited to measuring MAZ. Improved OCT resolution and other imaging modalities will facilitate a more detailed evaluation of the treatment site and local thermal damage.

In contrast, increased tissue penetration depth would also enhance

OCT's clinical utility and allow complete imaging of lesions that are thicker than 2 mm. For instance, OCT imaging of the deep boundary of a lesion prior to ablative laser de-bulking would allow dermatologists to choose the appropriate settings to precisely ablate the entire lesion and thus prevent reoccurrence.

OCT allows rapid high-resolution imaging of the skin and may serve as an adjunctive aid to help guide ablative laser setting selection by allowing real-time assessment before, during, and after laser ablative therapy. By imaging the target tissue before ablative laser therapy, OCT may enhance a laser surgeon's ability to determine the needed ablative depth to completely treat the lesion or skin condition.

By allowing visualization of the MAZ during ablative therapy, OCT may also aid laser surgeons in fine-tuning ablative laser settings and personalizing treatment to an individual patient's therapeutic needs. Imaging after ablative therapy would allow the laser surgeon to confirm that the entire lesion was removed by laser ablation.

From the description herein, it will be appreciated that the present disclosure encompasses multiple embodiments which include, but are not limited to, the following:

1. A system for performing laser therapy to a target tissue, comprising: (a) an imaging device; (b) a laser delivery device configured to apply laser radiation to a region of a target tissue; and (c) a system controller configured to receive input from the imaging device and generating an output to the laser delivery device controlling the settings of the laser delivery device.

2. The system of any preceding embodiment, wherein the imaging device is a device selected from the group of devices consisting of Optical Coherence Tomography (OCT), high frequency ultrasound (HF-US), Optical Coherence Microscopy (OCM) and Photoacoustic Microscopy (PAM) devices.

3. The system of any preceding embodiment, wherein the laser delivery device is a laser selected from the group of lasers consisting of an Erbium:YAG laser, a CO₂ laser, and a yttrium-scandium-gallium-garnet (Er:YSGG) laser.

4. The system of any preceding embodiment, wherein the controller comprises a circuit.

5. The system of any preceding embodiment, wherein the controller comprises: (a) a computer processor with an imaging interface with the imaging device and a laser interface with the laser delivery device; and (b) programming in a non-transitory computer readable medium and executable on the computer processor for performing steps comprising: (i) controlling the imaging device through the image interface to image a target tissue; (ii) analyzing acquired images of the target tissue; and (iii) designating laser parameters for treatment of target tissues.

6. The system of any preceding embodiment, the programming further performs the step comprising controlling the settings of the laser through the laser interface to have the designated laser parameters.

7. A system as recited in claim 1, further comprising a display, wherein images from the imaging device are displayed on the display.

8. The system of any preceding embodiment, further comprising: an image device user control interface; and a laser delivery device user control interface; wherein image acquisition, processing and target tissue identification are controlled by the image device user interface; and wherein laser device settings are calculated and configured by the laser device control interface.

9. The system of any preceding embodiment, further comprising: a common laser emitter imaging handpiece with one or more imaging heads, imaging sensors and laser beam emitters; wherein imaging scans are performed and laser beams delivered to a target tissue with the handpiece simultaneously or sequentially.

10. A system for performing laser therapy to a target tissue, comprising: (a) one or more imaging devices; (b) a laser delivery device configured to apply laser radiation to a region of a target tissue: (c) a computer processor operably coupled to the imaging device and the laser delivery device; and (d) programming in a non-transitory computer readable medium and executable on the computer processor for performing steps comprising: (i) acquiring from the imaging device an image of a region of tissue; (ii) identifying structures and boundaries of target tissue for treatment from acquired tissue images; (iii) determining laser type and laser device setting configuration for laser treatment based on the identified structure and boundary location of the target tissue; (iv) controlling the output to the laser delivery device with the determined setting configuration; and (v) performing laser treatment on at least a portion of the target region of tissue according to the configured laser treatment settings.

11. The system of any preceding embodiment, wherein the imaging device is a device selected from the group of devices consisting of Optical Coherence Tomography (OCT), high frequency ultrasound (HF-US), Optical Coherence Microscopy (OCM) and Photoacoustic Microscopy (PAM) devices.

12. The system of any preceding embodiment, wherein the laser delivery device is a laser selected from the group of lasers consisting of an Erbium:YAG laser, a CO₂ laser, and a yttrium-scandium-gallium-garnet (Er:YSGG) laser.

13. The system of any preceding embodiment, wherein the programming further performs the steps comprising: generating images from each imaging device; and displaying generated images and laser device configuration on a display.

14. A method for laser treatment of a tissue, the method comprising: (a) imaging a tissue with at least one imaging modality;(b) identifying target tissue boundaries from tissue images; (c) selecting a laser and laser settings based on tissue boundaries; and (d) performing laser treatment on at least a portion of the target tissue according to the selected laser treatment settings.

15. The method of any preceding embodiment, further comprising: re-imaging lasered target tissue intra-operatively to determine the extent of laser treatment on the target tissue; selecting laser settings based on images of target tissue; and performing a second laser treatment on at least a portion of the target tissue according to the selected laser treatment settings.

16. The method of any preceding embodiment, further comprising imaging lasered target tissue post operatively to confirm treatment results.

17. The method of any preceding embodiment, wherein the imaging modality is selected from the group of modalities consisting of Optical Coherence Tomography (OCT), high frequency ultrasound (HF-US), Optical Coherence Microscopy (OCM) and Photoacoustic Microscopy (PAM) devices or combinations thereof.

18. The method of any preceding embodiment, wherein the laser is selected from the group of lasers consisting of an Erbium:YAG laser, a CO₂ laser, and a yttrium-scandium-gallium-garnet (Er:YSGG) laser.

19. The method of any preceding embodiment, wherein the selection of laser settings comprises calculating laser fluence, spot size and dwell time to treat the target tissues to the identified boundaries.

20. The method of any preceding embodiment, wherein the selection of laser settings comprises: identifying the maximum ablative depth and width with the tissue images; and calculating laser fluence, spot size and dwell time to ablate the target tissues to the identified maximum ablative depth and width.

Embodiments of the present technology may be described with reference to flowchart illustrations of methods and systems according to embodiments of the technology, and/or algorithms, formulae, or other computational depictions, which may also be implemented as computer program products. In this regard, each block or step of a flowchart, and combinations of blocks (and/or steps) in a flowchart, algorithm, formula, or computational depiction can be implemented by various means, such as hardware, firmware, and/or software including one or more computer program instructions embodied in computer-readable program code logic. As will be appreciated, any such computer program instructions may be loaded onto a computer, including without limitation a general purpose computer or special purpose computer, or other programmable processing apparatus to produce a machine, such that the computer program instructions which execute on the computer or other programmable processing apparatus create means for implementing the functions specified in the block(s) of the flowchart(s).

Accordingly, blocks of the flowcharts, algorithms, formulae, or computational depictions support combinations of means for performing the specified functions, combinations of steps for performing the specified functions, and computer program instructions, such as embodied in computer-readable program code logic means, for performing the specified functions. It will also be understood that each block of the flowchart illustrations, algorithms, formulae, or computational depictions and combinations thereof described herein, can be implemented by special purpose hardware-based computer systems which perform the specified functions or steps, or combinations of special purpose hardware and computer-readable program code logic means.

Furthermore, these computer program instructions, such as embodied in computer-readable program code logic, may also be stored in a computer-readable memory that can direct a computer or other programmable processing apparatus to function in a particular manner, such that the instructions stored in the computer-readable memory produce an article of manufacture including instruction means which implement the function specified in the block(s) of the flowchart(s). The computer program instructions may also be loaded onto a computer or other programmable processing apparatus to cause a series of operational steps to be performed on the computer or other programmable processing apparatus to produce a computer-implemented process such that the instructions which execute on the computer or other programmable processing apparatus provide steps for implementing the functions specified in the block(s) of the flowchart(s), algorithm(s), formula(e), or computational depiction(s).

It will further be appreciated that the terms “programming” or “program executable” as used herein refer to one or more instructions that can be executed by a processor to perform a function as described herein. The instructions can be embodied in software, in firmware, or in a combination of software and firmware. The instructions can be stored local to the device in non-transitory media, or can be stored remotely such as on a server, or all or a portion of the instructions can be stored locally and remotely. Instructions stored remotely can be downloaded (pushed) to the device by user initiation, or automatically based on one or more factors. It will further be appreciated that as used herein, that the terms processor, computer processor, central processing unit (CPU), and computer are used synonymously to denote a device capable of executing the instructions and communicating with input/output interfaces and/or peripheral devices.

Although the description herein contains many details, these should not be construed as limiting the scope of the disclosure but as merely providing illustrations of some of the presently preferred embodiments. Therefore, it will be appreciated that the scope of the disclosure fully encompasses other embodiments which may become obvious to those skilled in the art.

In the claims, reference to an element in the singular is not intended to mean “one and only one” unless explicitly so stated, but rather “one or more.” All structural, chemical, and functional equivalents to the elements of the disclosed embodiments that are known to those of ordinary skill in the art are expressly incorporated herein by reference and are intended to be encompassed by the present claims. Furthermore, no element, component, or method step in the present disclosure is intended to be dedicated to the public regardless of whether the element, component, or method step is explicitly recited in the claims. No claim element herein is to be construed as a “means plus function” element unless the element is expressly recited using the phrase “means for.” No claim element herein is to be construed as a “step plus function” element unless the element is expressly recited using the phrase “step for.”

What is claimed is:

1. A system for performing laser therapy to a target tissue, comprising:
(a) an imaging device;
(b) a laser delivery device configured to apply laser radiation to a region of a target tissue; and
(c) a system controller configured to receive input from the imaging device and generating an output to the laser delivery device controlling the settings of the laser delivery device.

2. A system as recited in claim 1, wherein said imaging device is a device selected from the group of devices consisting of Optical Coherence Tomography (OCT), high frequency ultrasound (HF-US), Optical Coherence Microscopy (OCM) and Photoacoustic Microscopy (PAM) devices.

3. A system as recited in claim 1, wherein said laser delivery device is a laser selected from the group of lasers consisting of an Erbium:YAG laser, a CO₂ laser, and a yttrium-scandium-gallium-garnet (Er:YSGG) laser.

4. A system as recited in claim 1, wherein said controller comprises a circuit.

5. A system as recited in claim 1, wherein said controller comprises:
(a) a computer processor with an imaging interface with the imaging device and a laser interface with the laser delivery device; and
(b) programming in a non-transitory computer readable medium and executable on the computer processor for performing steps comprising:
(i) controlling the imaging device through the image interface to image a target tissue;
(ii) analyzing acquired images of the target tissue; and
(iii) designating laser parameters for treatment of target tissues.

6. A system as recited in claim 5, said programming further performs the step comprising controlling the settings of the laser through the laser interface to have the designated laser parameters.

7. A system as recited in claim 1, further comprising a display, wherein images from said imaging device are displayed on the display.

8. A system as recited in claim 1, further comprising:
an image device user control interface; and
a laser delivery device user control interface;
wherein image acquisition, processing and target tissue identification are controlled by the image device user interface; and
wherein laser device settings are calculated and configured by the laser device control interface.

9. A system as recited in claim 1, further comprising:
a common laser emitter imaging handpiece with one or more imaging heads, imaging sensors and laser beam emitters;
wherein imaging scans are performed and laser beams delivered to a target tissue with the handpiece simultaneously or sequentially.

10. A system for performing laser therapy to a target tissue, comprising:
(a) one or more imaging devices;
(b) a laser delivery device configured to apply laser radiation to a region of a target tissue;
(c) a computer processor operably coupled to the imaging device and the laser delivery device; and
(d) programming in a non-transitory computer readable medium and executable on the computer processor for performing steps comprising:
(i) acquiring from the imaging device an image of a region of tissue;
(ii) identifying structures and boundaries of target tissue for treatment from acquired tissue images;
(iii) determining laser type and laser device setting configuration for laser treatment based on the identified structure and boundary location of the target tissue;
(iv) controlling the output to the laser delivery device with the determined setting configuration; and
(v) performing laser treatment on at least a portion of the target region of tissue according to the configured laser treatment settings.

11. A system as recited in claim 10, wherein said imaging device is a device selected from the group of devices consisting of Optical Coherence Tomography (OCT), high frequency ultrasound (HF-US), Optical Coherence Microscopy (OCM) and Photoacoustic Microscopy (PAM) devices.

12. A system as recited in claim 10, wherein said laser delivery device is a laser selected from the group of lasers consisting of an Erbium:YAG laser, a CO₂ laser, and a yttrium-scandium-gallium-garnet (Er:YSGG) laser.

13. A system as recited in claim 10, wherein said programming further performs the steps comprising:
generating images from each imaging device; and
displaying generated images and laser device configuration on a display.

14. A method for laser treatment of a tissue, the method comprising:
(a) imaging a tissue with at least one imaging modality;
(b) identifying target tissue boundaries from tissue images;
(c) selecting a laser and laser settings based on tissue boundaries; and
(d) performing laser treatment on at least a portion of the target tissue according to the selected laser treatment settings.

15. A method as recited in claim 14, further comprising:
re-imaging lasered target tissue intra-operatively to determine the extent of laser treatment on the target tissue;
selecting laser settings based on images of target tissue; and
performing a second laser treatment on at least a portion of the target tissue according to the selected laser treatment settings.

16. A method as recited in claim 14, further comprising imaging lasered target tissue post operatively to confirm treatment results.

17. A method as recited in claim 14, wherein said imaging modality is selected from the group of modalities consisting of Optical Coherence Tomography (OCT), high frequency ultrasound (HF-US), Optical Coherence Microscopy (OCM) and Photoacoustic Microscopy (PAM) devices or combinations thereof.

18. A method as recited in claim 14, wherein said laser is selected from the group of lasers consisting of an Erbium:YAG laser, a CO₂ laser, and a yttrium-scandium-gallium-garnet (Er:YSGG) laser.

19. A method as recited in claim 14, wherein said selection of laser settings comprises calculating laser fluence, spot size and dwell time to treat the target tissues to the identified boundaries.

20. A method as recited in claim 14, wherein said selection of laser settings comprises:
identifying the maximum ablative depth and width with the tissue images; and
calculating laser fluence, spot size and dwell time to ablate the target tissues to the identified maximum ablative depth and width.
